(19) United States
(12) Patent Application Publication
(10) Pub. No.: US 2024/0245065 A1
(43) Pub. Date: Jul. 25, 2024

(54) BACTERIOPHAGES AND COMPOSITIONS THEREOF FOR CONTROLLING THE GROWTH OF PSEUDOMONAS LUNDENSIS IN MEAT PRODUCTS

(71) Applicants: PHAGELAB CHILE SPA, Santiago (CL); AGROSUPER COMERCIALIZADORA DE ALIMENTOS LTDA, Rancagua (CL)

(72) Inventors: Mauro CANAVAL ALFARO, Santiago (CL); Daniel TICHY NAVARRO, Santiago (CL); Matias Cristobal AGUILERA BARRIOS, Santiago (CL); Michael PINO BARRIENTOS, Santiago (CL); Rodrigo Andres NORAMBUENA VENEGAS, Santiago (CL); Maria Sofia ZAMUDIO CANAS, Santiago (CL); Pablo CIFUENTES PALMA, Santiago (CL); Hans PIERINGER CASTRO, Santiago (CL); Andrea Katherine LILLO SILVA, Rancagua (CL); Juan Carlos CARRENO PALMA, Rengo (CL)

(21) Appl. No.: 18/003,622

(22) PCT Filed: Aug. 11, 2022
(86) PCT No.: PCT/IB2022/057516

§ 371 Date: Dec. 28, 2022

(51) Int. Cl. A23B 4/22; A23L 3/3463; C12N 7/00

(52) U.S. Cl. CPC A23B 4/22; A23L 3/34635; C12N 7/00; C12N 2795/00021

(57) ABSTRACT

A formulation comprising a Pseudomonas-specific bacteriophage cocktail, useful for the prevention, reduction and/or treatment of infections caused by this bacterium through food contamination, particularly meat products. More specifically, the formulations for use in the food industry comprising at least one or more bacteriophages specific against Pseudomonas lundensis, where these are in combination with buffers, vehicles and/or acceptable excipients for the food industry.

### SPECIFICATION

The present invention refers to a formulation comprising a Pseudomonas-specific bacteriophage cocktail, useful for the prevention, reduction and/or treatment of infections caused by this bacterium through food contamination, particularly meat products. More the specifically, invention refers to formulations for use in the food industry comprising at least one or more bacteriophages specific against Pseudomonas lundensis, where these are in combination with buffers, vehicles and/or acceptable excipients for the food industry.

### FIELD OF INVENTION

The present invention refers to the food industry area. Particularly, the present invention refers to a formulation comprising a cocktail or mixture of bacteriophages, which exhibit lytic activity on Pseudomonas genus, in particular against Pseudomonas lundensis.

### BACKGROUND OF INVENTION

Highly perishable foods can be subject to contamination and become vehicles for the spread of foodborne diseases (FBD) after consumption (Heredia et al., 2014). Particularly, foods of meat origin are susceptible to contamination because after the slaughter of the animal, its tissues can be subject to contamination from various sources and also deterioration processes occur in the tissue due to the action of enzymes and chemical compounds (Heredia et al., 2014; Dave & Ghali, 2011). Furthermore, contamination of processed foods by microorganisms is mainly due to the susceptibility of these to become contaminated with pathogens their production stages (Heredia et al., 2014) Historically, foodborne disease outbreaks have been associated with the presence of microorganisms in meat products (Heredia et al., 2014).

There are several pathogens that affect foods in their production, packaging and transport. One of the bacteria that has been reported to be most prevalent in packaged food products such as meat are bacteria of the Pseudomonas genus.

It has been reported that Pseudomonas are more prevalent in meat packaged in modified atmosphere, especially in pork meat, with respect to vacuum-packed meat, their growth being favored by the 5° C. holding temperature. Furthermore, the growth rate of Pseudomonas spp. was reported to be considerably slow at 0° C., but increased at 2° C. and affected the shelf life of meat (Dave & Ghali, 2011) .

Pseudomonas spp. deteriorate food, particularly meat products due to their proteolytic, lipolytic, saccharolytic and biosurfactant capacities derived from the production of several enzymes. Where the latter, increase the availability of food for bacteria contributing to their growth (Morales, 2019) . Pseudomonas spp. presents a fast generation time under refrigeration being between 7.6 hours at 2° C. and 2.8 at 10° C. (Morales, 2019). Therefore, the rapid growth of these bacteria increases the spoilage of packaged foods especially if the cold chain is disturbed (Morales, 2019).

Pseudomonas lundensis has been specifically linked to spoilage of refrigerated meat. This bacterium is capable of forming biofilms at 30° C. but is much more efficient at temperatures between 4 and 10° C. (Molin, Goran, 1986). This bacterium produces a repertoire of different proteases and enzymes that act during refrigerated storage of meat, particularly red meat. The deterioration of meat causes the withdrawal, replacement and product loss, leading to significant economic losses for the industry.

Consequently, it is important and necessary to implement new technologies to increase the shelf life of foods, especially meat products during cold storage.

Among the usual strategies used for the decontamination of this type of food in the meat industry is the machinery and surfaces disinfection with chemical products in order to reduce the initial bacterial load in the meat. Generally, sodium hypochlorite and quaternary ammonium compounds are applied due to their low cost, although their use is controversial in some countries since they produce residues that can be toxic (Morales, 2019).

Initially, methods for preserving meat foods included salting, smoking, drying, fermentation and canning, all with the aim of preserving meat and preventing its deterioration (Dave & Ghali, 2011).

More recently, preservation of this type of food contemplates the application of synthetic preservatives among other compounds. However, the use of this type of preservatives is already being replaced by those of “natural” origin through the use of bacteria, antimicrobial peptides, natural extracts that antagonize pathogenic or unwanted bacteria. Also a way to maintain the shelf-life of meats is use of vacuum packaging media which slows the deterioration of these products (Morales, 2019).

However, these proposals are still indirect and the use of peptides is costly and difficult to access.

Other technologies have been described for extending the shelf-life of meat products, for example, the application of bacteriophages. Bacteriophages have been studied and used almost a century ago for disease prevention or treatment of numerous bacterial infections (Atterbury, 2009). In addition, the use of bacteriophages has received attention from the scientific and medical community, as they can be the replacement of conventional antibiotics because they suppress bacterial growth and prevent the emergence of resistant bacterial strains (Seo et al., 2016; Tanaka et al., 2018). In this regard, several investigations have been carried out where the use of bacteriophages for the control and treatment of pathogen growth in meat foods is evaluated.

Seo et al. (2016) addresses a study in which a specific bacteriophage that presented activity on E. coli O157:H7 in beef, chicken and pork was analyzed. The results of this study indicated that bacteriophage completely reduced growth of the bacteria within 10 minutes with a 10.000 MOI treatment of the bacteriophage. On the other hand, a 10.0000 MOI treatment of bacteriophage completely reduced 5 Log CFU/cm² of the bacteria in cattle and swine at 4 and 8 hours respectively.

Gordon and collaborators (2002) used bacteriophages for the control of Brochothrix thermosphacta bacterium, which causes bad odor in meats, on pork adipose tissue. While Galarcea and his team (2014) evaluated the control of Salmonella enteritidis using bacteriophages on smoked salmon samples.

US20160215273A1 discloses a bacteriophage composition, a probiotic bacteriophage and agents acceptable to the food industry. The bacteriophages of the composition exhibit lytic activity on Shigella spp. and are used for bacterial growth control in processed and unprocessed foods, wherein beef and pork are included, in addition to the use of this composition on surfaces in the food industry for cross-contamination prevention.

No specific solutions are available to reduce the bacterial load of Pseudomonas spp. in particular P. lundensis in refrigerated meat. The existing alternatives are scarce, not very effective, unsafe for human consumption and do not include as an alternative bacteriophages with specific lytic capacity in cold storage conditions of meat. It is important to generate an alternative that is safe, does not generate antibiotic resistance and acts specifically on P. lundensis.

### DESCRIPTION

The present invention relates to a formulation with antimicrobial activity, particularly antibacterial activity, comprising an effective amount of one or more specific bacteriophages with lytic activity against Pseudomonas spp. More specifically, the invention refers to the use of a bacteriophage formulation with antibacterial activity for the food industry comprising one or more bacteriophages specific against Pseudomonas genus together with buffers, vehicles or acceptable excipients. More particularly, the invention relates to an antibacterial formulation comprising bacteriophages specific against P. lundensis.

The bacteriophages comprising this formulation were deposited with the International Depositary Authority of Canada (IDAC), in accordance with the Budapest Treaty for the deposit of biological samples.

The bacteriophages that make up the formulation are identified as:
PluA-180A2 bacteriophage (deposit N° 121121-01)
P20P01 bacteriophage (deposit N° 220422-06)

The identification of the bacteriophages is established in their respective deposit certificate.

It is the object of the present invention to provide an antibacterial formulation to avoid the contamination of meat products by the appearance of pathogenic bacteria, preferably food products, when said food products are in their production chain and/or in cold storage.

It is the object of the present invention to provide an effective and safe antibacterial formulation for control of meat products contamination. These bacteriophages have not been previously described in the state of the art, so their use as a formulation and the application method are presented as a new alternative that fulfills the objective of solving the technical problem described.

The bacteriophages of the invention were isolated from samples of meat products and from the meat product processing chain, and were isolated for their specific effect against Pseudomonas spp.

It is part of the scope of the present invention the use of the antibacterial formulation for prevention and/or treatment of appearance of pathogenic bacteria in meat products during their storage stage. Particularly, when said pathogen corresponds to pathogens of Pseudomonas genus, in particular Pseudomonas lundensis. It is important to emphasize that the inventors have empirically determined that one of the most predominant bacterial genera in samples of meat products during storage and in the production chain is Pseudomonas, particularly Pseudomonas lundensis.

It is part of the scope of the present invention the use of the antibacterial formulation for the prevention and/or elimination of bacteria that contribute to the decomposition of meat in its cold storage stage. In particular, it is an object of the invention the use of the antibacterial formulation for preventing and/or treating and/or eliminating pathogenic bacteria of the Pseudomonas genus wherein said pathogenic bacteria is Pseudomonas lundensis.

It is part of the scope of the present invention to provide an antibacterial formulation that allows avoiding the emergence of antibiotic resistant strains, avoiding the use of chemical cleaning compounds and the residual accumulation thereof in the food industry, as is currently the case with conventional methods.

In one of the preferred embodiments of the invention, the antibacterial formulation is administered to the meat products in solid, powder, liquid, aqueous suspension, aerosol, emulsifier or other related form.

The bacteriophages described in the invention present favorable characteristics for use in food, such as meat products, and in human consumption.

The bacteriophages of the present invention meet the safety criteria for use in food products, i.e., they have no virulence factors, integrases, or antimicrobial resistance genes in their genome. The formulation comprises bacteriophages that meet the general guidelines for genomic information, which have been described by the Food and Drug Administration of the United States for use of a bacteriophage mixture approval (Phillipson et al., 2018).

The described characteristics allow establishing the use of these bacteriophages as safe for phage therapy.

As part of the scope of the invention, the bacteriophages are in a concentration of 1×10⁴-1×10⁸ PFU/mL, preferably 1×10⁵-1×10⁷ PFU/mL. In a preferred embodiment, the bacteriophages are at a concentration of 1×10⁵-1×10⁷ PFU/mL wherein said bacteriophages are at equal or different concentrations as corresponds to a mixture of one or more bacteriophages.

As part of the present invention, in the formulation, the buffer, vehicle and excipients are those acceptable in food industry, particularly in meat products. As part of the present invention, the bacteriophages, in relation to the other products of the formulation (buffer, vehicle and excipients acceptable in the food industry) are in a ratio of 1:1000 to 1000:1.

In the present invention, vehicles, excipients and buffers are compatible with oral administration. Examples of buffer solutions are TBS, PBS, SM, among others.

Acceptable vehicles, buffers or excipients are selected from the group comprising sodium chloride, EDTA, sodium 4-hydroxybenzoate, water or any other acceptable excipient in the food industry and specifically for administration in meat products.

As part of the present invention, it is considered as an additional object to provide a method for the prevention and/or treatment of meat products contamination caused by Pseudomonas spp. wherein said method comprises administering or placing in contact the described antibacterial formulation with the meat product. In particular, the administration of the antibacterial formulation is carried out in packaged meat products that must also be transported for subsequent sale and consumption. Said administration method comprises bringing the antibacterial formulation in contact with the meat product.

In one of the preferred embodiments, the administration or contacting of the bacteriophage-comprising formulation with the meat product is carried out prior to the packaging of the product.

The inventors demonstrated that different bacterial genera predominate in samples of meat products during storage under cold conditions. In particular, bacteria of the Pseudomonasgenus stand out for their high predominance. In particular, it was determined by means of sequencing, bioinformatic analysis and bacterial culture that P. lundensis is predominant in samples of meat products. This bacterium is associated with putrefaction and decomposition of meat.

In the present invention, the administration of the antibacterial formulation on meat product samples decreases the bacterial count by 1 to 4 orders of magnitude, preferably by 1 to 2 orders of magnitude after a 8 days period. The bacteriophages of the present invention can reduce the bacterial load of P. lundensis.

In other preferred embodiments of the invention, the formulation has an antibacterial effect on meat products samples, particularly pork. The administration or contact of the formulation with the meat allows the significant decrease of up to 2 log CFU/reaction of bacteria, particularly against P. lundensis at day 8.

As described, the antibacterial formulation of the present invention is useful for prevention of growth of Pseudomonas genus bacterial strains in food, in particular for preventing and decreasing the growth of P. lundensis in meat products. The formulation is useful to avoid the appearance of antibiotic resistant strains, avoid the use of chemical cleaning compounds and their residual accumulation.

It is part of the scope of the invention, a method for preventing contamination of packaged meat products by Pseudomonas spp. comprising the administration of the described formulation, particularly in meat products.

By means of this method, on day 1 of administration, P. lundensis count is decreased by one order of magnitude. By means of the present method, on day 8 post-administration, P. lundensis count is decreased by two orders of magnitude.

### DEFINITIONS

The following are definitions that will allow a better understanding of the present invention. The terms indicated correspond to scientific definitions.

The term “antimicrobial agent” corresponds to any substance, solution, chemical and/or biological compound that allows the destruction of microorganisms or the prevention of their proliferation. For the present invention the term “antimicrobial” comprises the total elimination or growth control of a particular bacterial population.

The term “antimicrobial activity” refers to the characteristic of a substance, solution, chemical and/or biological compound to eliminate or reduce the bacterial population growth.

The term “bacteriophage” or “phage” refers to a type of virus that has the ability to infect and use specific cells, in this case specific bacteria, as a host. In the present invention the term “bacteriophage” is used to indicate those phages which are specific to infect or use as a host bacterial cells.

The term “lytic activity” refers to a bacteriophages characteristic in which they multiply inside a host cell, in this case a bacterium, to then destroy it by cell lysis and release the phage progeny into the medium and be able to use other bacteria as a host.

When the term “cocktail” is used, it refers to a mix or mixture of one or more bacteriophages that exhibit antimicrobial properties, particularly antibacterial.

The term “microbiological count” includes any microbiological technique that makes it possible to count or determine the number of microorganisms present in a sample.

The term “MAC” or “mesophilic aerobic count” corresponds to a microbiology technique that allows the determination of the number of total microorganisms present in a sample that are characteristically aerobic or facultative anaerobic, mesophilic or psychrophilic and capable of growing in culture media.

The term “CFU/mL” or “CFU/g” stands for Colony Forming Units per milliliter or gram of composition and indicates the number of live microorganisms capable of forming colonies in a liquid medium in a given volume or mass.

The term “PFU/mL” stands for lysis Plate-Forming Units per milliliter of the composition, it allows to quantify the phage viral particles capable of lysing or breaking cells in a given volume. It indicates the number of lysis halos present on a bacterial culture plate per unit volume of virus, where each lysis halo is formed (theoretically) by the presence of a single virus.

The term “order of magnitude” refers to the value of a number that is raised in 10 base when expressing a number in scientific notation.

When reference is made to the term “qPCR” or “real-time PCR”, it refers to a variant of PCR or Polymerase Chain Reaction technique but in this case is quantitative, i.e. it simultaneously and absolutely amplifies and quantifies the DNA amplification product.

When reference is made to the term “acceptable vehicles, buffers and/or excipients” it refers to any component, regardless of its nature, that allows the correct administration of the bacteriophages in the subject to be treated. In the case of the present invention they correspond to components for use in meat products.

### DESCRIPTION OF FIGURES

FIG. 1. Microbiological count in A-PSLU bacteriophage cocktail efficacy assay in pork rib samples. The graph shows the bacterial growth (CFU/g meat) in pork rib cuts samples infected with P. lundensis (Pslu 040). The bacterial growth of each of the samples analyzed is shown in the graph as follows: (•) indicates pork rib samples infected with P. lundensis without treatment; (▪) indicates pork rib samples infected with P. lundensis with A-PSLU bacteriophage cocktail treatment at a concentration of 10⁷ PFU/mL; (⋄) indicates pork rib samples infected with P. lundensis with A-PSLU bacteriophage cocktail treatment at a concentration of 10⁶ PFU/mL; (♦) indicates uninfected and untreated pork rib samples, and (○) indicates pork rib samples uninfected but treated with 10⁷ PFU/mL A-PSLU bacteriophage cocktail. Error bars correspond to SEM.

FIG. 2. Absolute quantification of P. lundensis in pork rib samples. The graph shows the detection of P. lundensis 040 transcripts by qPCR on day 0 to day 8 of sampling. The quantification of P. lundensis transcript is indicated as follows: (•) indicates pork rib samples infected with P. lundensis without treatment; (▪) indicates pork rib samples infected with P. lundensis with A-PSLU bacteriophage cocktail treatment at a concentration of 10⁷ PFU/mL; (⋄) indicates pork rib samples infected with P. lundensis with A-PSLU bacteriophage cocktail treatment at a concentration of 10⁶ PFU/mL; (♦) indicates uninfected and untreated pork rib samples, and (o) indicates pork rib samples uninfected but treated with 10⁷ PFU/mL A-PSLU bacteriophage cocktail. Error bars correspond to SEM post Dunnett's statistical test.

### EXAMPLES

### Example 1. Determination of the Predominant Microorganisms Responsible for the Spoilage of Meat Products

### Obtaining Samples for Microbiome Analysis

A microbiome analysis of meat samples from domestic market and samples from international market was carried out to determine the predominant microorganisms in these products. For this purpose, 50 pork loin samples, 50 pork belly samples, 40 pork rib samples and 40 leg pulp samples were taken. All the samples were stored under vacuum, emulating the storage conditions of the meat industry where refrigeration temperatures between −5° C. and 4° C. are used.

Ten (10) replicates of each sample were processed, cut and washed under sterile conditions, and then the microorganisms present in the samples were sedimented in pellets.

The samples from the national market were stored for a period of 25 days for analysis, while the samples from the international market were stored for a maximum of 70 days.

### Microbiome Analysis of Meat Samples

The presence of bacteria in the pellets was corroborated by amplification of DNA coding for the 16S ribosomal subunit. Total DNA content was purified using the DNeasy PowerFood Microbial kit (Qiagen), following the manufacturer's instructions. Double-stranded DNA libraries were prepared from the purified DNA and library quality was confirmed using Qubit Fluorometer 3.0 (Thermo Fisher) and Fragment Analyzer (Agilent) according to the manufacturer's instructions.

Genomic libraries were subjected to massively parallel sequencing of the DNA coding for the 16S ribosomal subunit of the organisms present in the microbiome.

The results of these analyses indicate the microorganisms present in the national and international product samples (Table 1), with a predominance of bacteria of the Pseudomonas genus.

TABLE 1
Microorganisms present in meat samples from
national and international markets.

| National market samples | | International market samples | |
|---|---|---|---|
| Product | Microorganisms | Product | Microorganisms |
| Vetoed pork | Pseudomonas spp. | Pork loin | Pseudomonas |
| rib | Psycrobacter spp. | | psychrophila. |
| | Pseudomonas | | Carnobacterium |
| | psychrophila. | | spp. |
| | Enterobacterias | | |
| Leg pulp | Pseudomonas | Pork belly | Pseudomonas spp. |
| | psychrophila. | | Pseudomonas |
| | Carnobacterium | | psychrophila. |
| | spp. | | Carnobacterium |
| | Serratia spp. | | spp. |

### Obtaining of Strains and Identification of Isolated Bacteria

The liquid obtained in the sample washing process was also seeded on non-selective agar and incubated to isolate colonies. From each isolate, libraries were generated and 16S subunit DNA sequencing was performed by Sanger method.

A total of 220 bacteria were isolated from each product, of which 187 isolates were identified to the species or genus level. Through a pairwise alignment algorithm, a total of 10 different clusters were identified, consisting of microorganisms sharing sequences with 100% identity and lengths between 340 and 380 nucleotides.

Results of the bioinformatic analysis of the sequencing information allow us to conclude that different clusters of predominant microorganisms were identified in meat samples, being predominant bacteria of the genera Carnobacterium, Aeromonas salmoncida, Yersinia spp., Yersinia nurmii and Pseudomonas lundensis.

In case of Pseudomonas, it was possible to isolate Pseudomonas such as P. lundensis and P. fluorescens, with P. lundensis being predominant. These are Gram-negative bacteria that grow under aerobic conditions. Both detected species have been reported as bacteria involved in meat spoilage (Dave y Ghaly, 2011; Lee et al., 2017; Nychas et al., 2008).

### Example 2. Bacteriophages Characterization

Samples collection for bacteriophages purification.

In order to obtain bacteriophages that could have an effect on the obtained bacteria, samples were taken from work surfaces in the meat industry. For this purpose, Stuart-InT transport torulas were used. Solid and liquid biological samples were also taken in 50 ml conical tubes.

### Bacteriophages Purification

From the samples obtained, viral particles content was purified. Bacteriophages were isolated by selecting those with suitable qualitative bacterial activity profiles and their functional titer was determined.

Candidate bacteriophages effect on bacterial growth kinetics based on continuous monitoring of bacterial growth was analyzed using the bacteria selected in previous example. In particular, bacteria of different species were used to search for bacteriophages (Table 2).

TABLE 2
Bacteria used for bacteriophages isolation.

| | No of isolated bacteria | | | | |
|---|---|---|---|---|---|
| Bacteria | PULP | CUTLET | LOIN | BELLY | TOTAL |
| Carnobacterium spp | 10 | 10 | 11 | 35 | 66 |
| Carnobacterium | 12 | 0 | 23 | 15 | 50 |
| Aeromonas salmoncida | 26 | 4 | 0 | 0 | 30 |
| Yersinia spp. | 2 | 16 | 0 | 0 | 18 |
| Yersinia nurmii | 1 | 4 | 0 | 4 | 9 |
| Pseudomonas spp | 0 | 7 | 0 | 0 | 7 |
| Pseudomonas lundensis | 0 | 4 | 0 | 0 | 4 |
| TOTAL | 51 | 45 | 34 | 54 | 184 |

From the bacteriophages antibacterial activity analysis according to the lysis plaques formation (lysis plaque forming units or PFUs) when confronted or co-incubated with the isolated bacteria, 21 viral particles mixture (VPM) were obtained that showed activity against Carnobacterium spp, Carnobacterium divergens and Pseudomonas lundensis. (Table 3).

TABLE 3
Viral particles mixture (VPM) that showed antibacterial activity.

| Bacteria of | No of VPM | Type of | |
|---|---|---|---|
| interest | with activity | activity | Source |
| Carnobacterium | 13 | Partial lysis | Drainage, |
| spp | | | animal tissue. |
| Carnobacterium | 7 | Partial lysis | Animal tissue, |
| | | | cutting tools. |
| Pseudomonas | 2 | Total lysis | Brine. |
| lundensis | | | |

With this result, the VPM showing total lysis was selected and tested for antibacterial activity against Pseudomonas spp by double-layer agar plate assay.

Thus, the two VPMs identified that showed activity against Pseudomonas lundensis were purified by routine microbiological techniques. One of the purified bacteriophages was named PLuA-180A2 and the other was named P20P01. The cocktail comprising both bacteriophages was termed A-PSLU, and corresponds to the cocktail that was evaluated in the remaining preferred examples shown in the present invention.

### Example 3. Efficacy Evaluation Trial: Preparation of Meat Samples and Samples Inoculation With P. lundensis

### Matrix Preparation

From freshly slaughtered pork rib cuts, 3 cuts were taken which were divided into 24 parts (72 samples in total), each part presented a mass of 10 g or dimensions of 10×10×5 mm approximately. Before submitting these samples to the A-PSLU bacteriophage cocktail, they were sterilized by immersion in 70% ethanol, dried for 10 minutes in a biosafety cabinet 2, then the samples were immersed in sterile water, dried for 10 minutes and finally incubated at 20° C. for 18 hours. Finally, the samples were stored in 6-well plastic plates awaiting the next assay (Spricigo et al., 2013; Seo et al., 2016).

Pork Samples Infection With P. lundensis

Samples were divided into two analysis groups. The first group corresponds to samples “infected” with P. lundensis. In this case, 12 pork rib samples were inoculated with 100 μl of a culture of P. lundensis 040 with a concentration of 1×10⁵ CFU/mL, 0.1% buffered pepton water. The inoculum was homogeneously distributed over 12 samples using a sterile loop and allowed to dry in a laminar flow cabinet for 30 minutes so that the bacteria could adhere to the matrix.

The second group corresponds to “uninfected” samples, where 12 meat samples were treated only with 0.1% sterile peptonized water.

### Example 4. Efficacy Evaluation Trial: Sampling of P. lundensis Load in Pork Ribs After Bacteriophages Treatment

Four pork rib samples were taken from the “infected” group and four pork rib samples from the “uninfected” group.

Pork rib samples infected with P. lundensis Pslu 040 were immersed for 20 seconds in saline solution or in bacteriophage cocktail, the latter being classified as “high dose” and “low dose” (Table 1). The samples were then dried for 10 minutes in a biosafety cabinet 2, to finally store the samples in a 6-well plate at 4° C.

The cocktail A-PSLU comprises a combination of equal parts of the following bacteriophages:

Bacteriophage PluA-180A2 (deposit N° 121121-01)

Bacteriophage P20P01 (deposit N° 220422-06)

TABLE 4
Experimental groups for the ex vivo efficacy
test of bacteriophage treatment on pork ribs.

| Group | Treatment |
|---|---|
| High dose | “A-PSLU” cocktail - 10⁷ UFP/mL |
| Low dose | “A-PSLU” cocktail - 10⁶ UFP/mL |
| No treatment | Sterile saline solution (NaCl 0.9%) |

In addition to differentiating the samples into “high dose” and “low dose” treatments, a table was made in which it is possible to observe the number of analyses performed for each treatment, replicates, days of analysis and total number of analyses for the pork rib samples (Table 5).

TABLE 5
Number of analyses performed on pork rib samples.

Replicates

| | | |
|---|---|---|
| Group | Day 1 | Day 8 |

| | | | |
|---|---|---|---|
| High dose | Infected | 3 | 3 |
| | Uninfected | 2 | 2 |
| Low dose | Infected | 3 | 3 |
| No | Infected | 3 | 3 |
| treatment | Uninfected | 3 | 3 |

| | | |
|---|---|---|
| Subtotal per day | 14 | 14 |

| | |
|---|---|
| Total pork ribs | 28 analyses |

### Sampling and Processing of Pork Rib Samples

Samples were mechanically homogenized for 1 minute, flesh tissue and eukaryotic cell debris were removed and bacteria were recovered. The homogenized samples were resuspended in 4 volumes of TPA inside a sterile plastic bag. From the homogenized sample in the bag, 35 mL were taken and centrifuged at 600 g for 5 minutes, after centrifugation the pellet was discarded. The supernatant obtained was centrifuged again, but at 3250 g for 10 minutes, this time the pellet was retained. The pellet was resuspended in 250 μL of 0.1% APT. These samples were divided to use 50 μL for the microbiological counting assay and 200 μL for absolute quantification assay by qPCR, where the latter was centrifuged at 5000 g for 10 minutes and the pellet was stored at −20° C. until use.

### Example 5. Efficacy Evaluation Trial: Microbiological Counting and Molecular Analysis of Pork Rib Samples

### Microbiological Counting of Samples

Pellet samples were analyzed by mesophilic aerobic count (MAC). In this assay, the pellets were resuspended in 250 μL of 0.1% TPA and 8 serial dilutions were prepared in a 1:10 ratio. 10 μL of each dilution was seeded on TSA plates (with previously labeled quadrants) and incubated at 30° C. for 18 hours. Microbiological titer was determined in CFU/g by counting colonies in each quadrant.

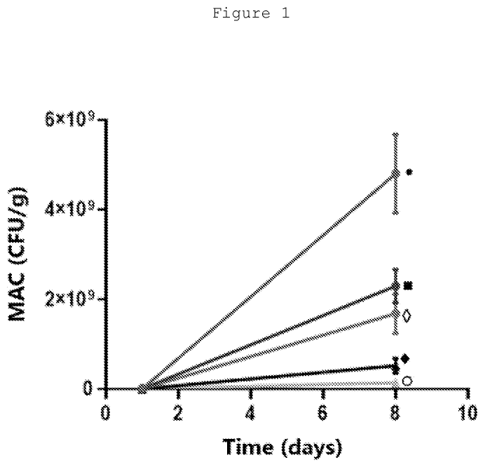

The results of the microbiological titer counting assay indicate that for days 1 and 8 of analysis the “non-infected” groups have a lower titer compared to the group “infected” with P. lundensis (FIG. 1), corroborating infection where applicable.

It was also observed that on day 1 in infected meat, administration of bacteriophage treatment at a concentration of 10⁶ PFU/mL (+), decreased P. lundensis counts by almost an order of magnitude.

As the test days progressed, the titer of each experimental group increased between 5.22 and 6.45 logarithms in 7 days (Table 6). The largest increases correspond to the untreated groups, while the treated groups increase at a lower rate.

By day 8 of testing all sampling groups exceeded the allowable CFU/g limit, however, the uninfected group treated with the high-dose cocktail showed a decrease of 0.88 log CFU/g compared to the untreated group. This may be attributed to the bactericidal activity of the cocktail on the uninfected samples.

TABLE 6
Increase in the microbiological titer in the pork rib samples.

| | | | | | Increase |
|---|---|---|---|---|---|
| | Day 1 | | Day 8 | | from day 1 |
| | Titer | Log | Titer | Log | to day 8 |
| Assay | (UFC/g) | (UFC/g) | (UFC/g) | (UFC/g) | Log (UFC/g) |
| Uninfected; | 2.57 × 10² | 2.41 | 5.13 × 10⁸ | 8.71 | 6.30 |
| Untreated | | | | | |
| Uninfected; | 8.32 × 10² | 2.92 | 1.35 × 10⁸ | 8.13 | 5.22 |
| Cocktail | | | | | |
| (1E7 | | | | | |
| PFU/mL) | | | | | |
| Infected | 1.48 × 10³ | 3.17 | 4.2 × 10¹¹ | 9.62 | 6.45 |
| (Pslu_040); | | | | | |
| Untreated | | | | | |
| Infected | 1.62 × 10³ | 3.21 | 1.66 × 10¹¹ | 9.22 | 6.02 |
| (Pslu_040); | | | | | |
| Cocktail | | | | | |
| (1E6 | | | | | |
| PFU/mL) | | | | | |
| Infected | 1.4 × 10³ | 3.15 | 2.09 × 10¹¹ | 9.32 | 6.17 |
| (Pslu_040); | | | | | |
| Cocktail | | | | | |
| (1E7 | | | | | |
| PFU/mL) | | | | | |

Absolute Quantification of Pork Samples by qPCR

Absolute quantification of P. lundensis in pork rib pellet samples was performed by qPCR using specific primers.

The sample pellet was resuspended in 100 μL of 7.5% Chelex (Biorad simple DNA extraction medium) and incubated at 56° C. for 30 minutes, then the incubation temperature was increased to 100° C. for 8 minutes, and the supernatant was recovered. Serial dilutions were performed in ultrapure water at a ratio of 1:1000 for each sample.

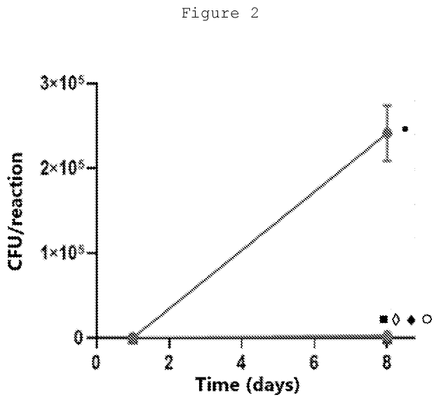

Results of the absolute quantification of pellets obtained from pork rib samples show that P. lundensis transcript levels at day 0 are below the level of detection limit for uninfected samples except for the infected pork rib sample that was treated with the bacteriophage cocktail at a concentration of 10⁷ PFU/mL, which did show detection of P. lundensis transcripts (FIG. 2).

Results of absolute quantification in pork rib samples allow establishing significant differences at day 8 of the test, since a decrease of up to 2 log CFU/reaction of P. lundensis bacteria was observed in the groups treated with bacteriophage cocktail (infected and uninfected samples) (FIG. 2).

### Bibliografía

Dave D, Ghaly A. (2011). Meat spoilage mechanisms and preservation techniques: a critical review. American Journal of Agricultural and Biological Sciences, 6(4), 486-510.

Galarcea N, Bravo J, Robeson J, Borie C. (2014). Bacteriophage cocktail reduces Salmonella enterica serovar Enteritidis counts in raw and smoked salmon tissues. Rev Argent Microbiol. 2014; 46 (4): 333-337.

Gordon Greer, Bryan D Dilts. (2002). Control of Brochothrix thermosphacta spoilage of pork adipose tissue using bacteriophages. J Food Prot. 65 (5): 861-3.

Heredia N, Davila-Aviña J, Solis L, Garcia S. (2014). Meat products: main pathogens and non-thermal control strategies. NACAMEH Vol. 8, Sup. 1, pp. S20-S42, 2014.

Molin, Goran TERNSTROM, A. & URSING, J. (1986). Notes: Pseudomonas lundensis, a New Bacterial Species Isolated from Meat. International Journal of Systematic Bacteriology. 36. 10.1099/00207713-36-2-339.

Morales P. (2019). “Evaluación de la biodiversidad de Pseudomonas alterantes de pollos refrigerados y su sensibilidad frente a Lactobacillus, potenciales bio-preservantes de la carne de ave. Tesis para la obtención del grado de Doctor. Universidad de Chile.

R. J. Atterbury. (2009). Bacteriophage biocontrol in animals and meat products. Microb Biotechnol. 2(6): 601-612.

Seo J, Seo D J, Oh H, Jeon S B, Oh M H, Choi C. 2016. Inhibiting the Growth of Escherichia coli O157: H7 in Beef, Pork, and Chicken

Meat using a Bacteriophage. Korean J Food Sci Anim Resour. 36 (2): 186-193.

Spricigo D, Bardina C, Cortés P, Llagostera M. (2013). Use of a bacteriophage cocktail to control Salmonella in food and the food industry. International journal of food microbiology, 165 (2), 169-174.

Tanaka C, Yamada K, Takeuchi H, Inokuchi Y, Kashiwagi A, Toba T. (2018). A Lytic Bacteriophage for Controlling Pseudomonas lactis in Raw Cow's Milk. Appl Environ Microbiol; 84 (18).

1. An antibacterial formulation comprising bacteriophages exhibiting lytic activity against strains of Pseudomonas spp, wherein the formulation comprises:
a) an effective amount of the at least one of the bacteriophages:
PluA-180A2 bacteriophage (deposit N° 121121-01)
P20P01 bacteriophage (deposit N° 220422-06), and
b) a buffer, a vehicle and/or acceptable excipients.

2. An antibacterial formulation comprising a mixture of bacteriophages exhibiting lytic activity against strains of Pseudomonas spp, wherein the formulation comprises:
a) an effective amount of the followings bacteriophages:
PluA-180A2 bacteriophage (deposit N° 121121-01)
P20P01 bacteriophage (deposit N° 220422-06), and
b) a buffer, a vehicle and/or acceptable excipients.

3. The antibacterial formulation according to claim 1, wherein the bacteriophages are in concentrations of 1×10⁴ to 1×10⁸ UFP/mL.

4. The antibacterial formulation according to claim 3, wherein the bacteriophages are in concentrations of 1×10⁵ to 1×10⁷ UFP/mL.

5. The antibacterial formulation according to claims 1, wherein the formulation corresponds to a formulation for the food industry.

6. The antibacterial formulation according to claim 5, wherein the formulation is administered as a solid, powder, liquid, aqueous suspension, aerosol, emulsifier, or other related forms.

7. A method for prevention of growth of bacterial strains of the Pseudomonas genus in food comprising applying the antibacterial formulation according to claim 1 to the food.

8. The method according to claim 7, wherein the bacterial strains of the Pseudomonas genus correspond to Pseudomonas lundensis.

9. The method according to claim 7, wherein the food corresponds to meat products.

10. The method according to claim 7, wherein the formulation prevents the emergence of antibiotic resistant strains in meat products.

11. A method for preventing and eliminating contamination of meat products by Pseudomona spp. comprising administering the formulation according to claim 1.

12. The method for preventing and eliminating contamination of meat according to claim 11, wherein on day 1 of administration, P. lundensis count is decreased by an order of magnitude.

13. The method for preventing and eliminating contamination of meat according to claim 11, wherein on day 8 post administration, P. lundensis count is decreased by two orders of magnitude.

14. The antibacterial formulation according to claim 2, wherein the bacteriophages are in concentrations of 1×10⁴ to 1×10⁸ UFP/mL.

15. The antibacterial formulation according to claim 14, wherein the bacteriophages are in concentrations of 1×10⁵ to 1×10⁷ UFP/mL.

16. The antibacterial formulation according to claim 2, wherein the formulation corresponds to a formulation for the food industry and wherein the formulation is administered as a solid, powder, liquid, aqueous suspension, aerosol, emulsifier, or other related forms.

17. A method for prevention of growth of bacterial strains of the Pseudomonas genus in food comprising applying the antibacterial formulation according to claim 2 to the food.

18. The method according to claim 17, wherein the bacterial strains of the Pseudomonas genus correspond to Pseudomonas lundensis and wherein the food corresponds to meat products.

19. The method according to claim 17, wherein the formulation prevents the emergence of antibiotic resistant strains in meat products.

20. A method for preventing and eliminating contamination of meat products by Pseudomona spp. comprising administering the formulation according to claim 2.
